(19) United States
(12) Patent Application Publication
(10) Pub. No.: US 2010/0095407 A1
(43) Pub. Date: Apr. 15, 2010

(54) PAC 941

(75) Inventor: Jorge Pinochet, Barcelona (ES)

Correspondence Address: MARK D MILLER;KIMBLE, MACMICHAEL & UPTON, 5260 NORTH PALM AVENUE, SUITE 221, FRESNO, CA, 93704, US

(73) Assignee: AGROMILLORA IBERIA, S.L., Barcelona (ES)

(21) Appl. No.: 12/250,738

(22) Filed: Oct. 14, 2008

(51) Int. Cl. A01H 5/00

(52) U.S. Cl. 800/298

(57) ABSTRACT

A new and distinct plum almond hybrid cultivar for rootstock use that originated as an open pollinated seedling as provided. This new invention (cultivar) is particularly well suited for use as a plum, peach, nectarine, apricot and almond rootstock. It is found that this new invention (cultivar) exhibits good root anchorage as well as a significantly reduced tendency to form unwanted suckers.

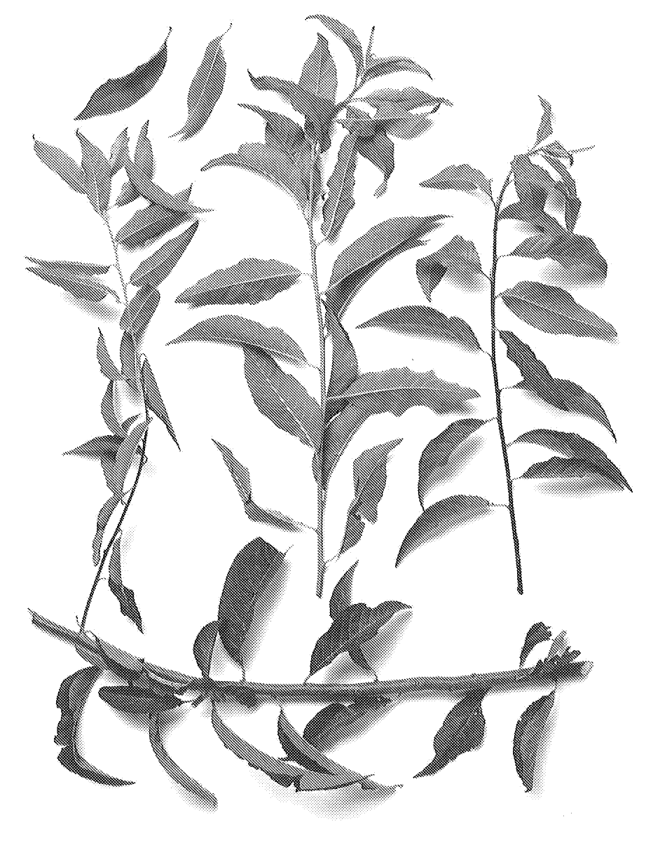

### BACKGROUND OF THE NEW VARIETY

The original hybrid was discovered in Zaragoza, Spain and is thought to be an open pollinated hybrid of the Myrobalan plum (Prunus cerasifera) and almond (Prunus dulcis), both of unknown origin. Several original clones were found in a nursery in 1994 in Sabiñan, Zaragoza, observed and tested for nematodes and ease of propagation by wood cuttings resulting in the selection of one single clone, PAC 941, which became the experimental designation of the selection and afterwards the new cultivar.

### SUMMARY OF THE INVENTION

In 1994, several seedlings having a similar growth pattern and morphology were discovered in a plum nursery in Sabiñan, Zaragoza, Spain. They differed in morphology at the same site and are thought to be an open pollinated interspecific hybrid between plum and peach or plum and almond. The source of the seed that resulted in the production of the new cultivar of the present invention is not known but is thought to be local. Seven similar and distinct seedlings were observed propagated by cuttings in 1995 and evaluated against root-knot nematodes (Meloidogyne species) and lesion nematodes (Pratylenchus vulnus) in 1996. The most interesting clone from the nematode standpoint was chosen for further study. The rest were discarded. The selected clone was designated PAC 941. In 1999, PAC 941 was introduced into the United States through NRSP/IR2, Washington State University in Prosser, Wash. as Plum PAC 941.

PAC 941 was propagated in vitro from the original mother plant and evaluated for biotic and abiotic stress, and tested in field trials between 1997 and 2007 in Spain. It was found to possess horticultural characteristics (discussed hereinafter) that were superior to widely grown similar plum rootstocks such as ‘Marianna 2624’, ‘Myrobalan 29C’, ‘Marianna GF-81’ and ‘Adesto 101’.

The new plum almond hybrid cultivar of the present invention was found to exhibit the following: (1) excellent root anchorage when compared to other peach and plum rootstocks; (2) a substantially less propensity to form suckers when compared to other plum rootstocks; (3) aptitude to serve as a plum, nectarine, peach, almond or apricot rootstock; and (4) excellent adaptation to limited soil conditions, especially water logging and calcareous soils (high lime content).

The root system extends more deeply and more vertically into the ground than ‘Marianna 2624’ and ‘Adesto 101’. The lesser propensity to form suckers offers significant savings in labor. Its specter of compatibility with several Prunus species used as commercial varieties, mainly, peach, plum nectarine, almond and apricot, makes this cultivar a most interesting choice as a rootstock in sites where soil limitations do not allow, for example, peach cultivation with traditional peach or peach-almond rootstocks. Its outstanding adaptation to poor, heavy soils with high lime content (that cause iron chlorosis), offers a better production alternative for several Prunus species in limiting conditions and with a cost reduction in iron kelate applications. These characteristics make this cultivar ideal for replant situations. When compared with ‘Marianna 2624’ or ‘Myrobalan 29C’, the new cultivar does not form fruit.

The new cultivar is most probably a standard diploid as are both Prunus cerasifera and Prunus dulcis as parents. The new cultivar has served well when grafted with the French prune variety ‘Stanley’ and Japanese plum (Prunus salicina) varieties ‘Black Gold’, ‘Red Beaute’, ‘Golden Globe’, ‘Grand Rosa’, and ‘Son Gold’. The new cultivar has shown good compatibility with the nectarine (Prunus persica) varieties ‘Summergrand’, ‘Big Top’, and ‘Alexandra’ as well as with the almond varieties (Prunus dulcis) ‘Moncayo’, ‘Desmayo Largueta’, ‘Butte’, ‘Nonpareil’ and ‘Monterey’.

The new cultivar (invention) has been reproduced at a commercial nursery, North American Plants, located in McMinnville, Oreg. by in vitro techniques on several occasions. Such propagations have confirmed that the characteristics of the new cultivar are stable and are reliably transmitted to subsequent generations.

### BRIEF DESCRIPTION OF THE PHOTOGRAPH

The accompanying colored photograph displays terminal leaf tips along with mature leaves, both upper and lower surfaces. In addition, a section of older branch material is displayed. The trees of the new cultivar were grown at Sun World, near Bakersfield, Calif. Color designations are presented with reference to the “Dictionary of Color” by Maerz and Paul, First Edition (1930).

### DETAILED DESCRIPTION

### Tree

### Size:

Height: 4-5 feet (121.92-152.4 cm)

Width: 4-5 feet (121.92-152.4 cm)

Figure: Upright in form in open vase system

Vigor: very good for 2^{nd} year
Hardiness: hardy under climatic conditions in the Central San Joaquin Valley of California
Productivity: none
Bearing: non bearing

### Trunk

Size: medium

Circumference: 4.5 inches (11.43 cm), at 8 inches (20.32 cm) above ground level

Texture: slightly rough

### Color: Teakwood Pinecone+(15-C-9)

Lenticels; moderate in number

Size: 1.5-2.5 inches (3.81-6.35 cm)

### Branches

Size: medium

Size over one year old: 2.5 inches (6.85 cm) at 10 inches (25.4 cm) above crotch

Crotch angle: approximately 32°

Texture: nearly smooth

### Color: Whippet (15-L-10)

Current Season Piquant Gr. (20-K-6)

Internode Length: 1.5-2.5 inches (3.81-6.35 cm)

Color as internodes mature: Caldera (7-L-12)

### Leaves

Size: small to medium

Length: 2.59-3.54 inches (6.58-8.99 cm)

Width: 1.15 inch (2.92 cm)

Form: ovate to lanceolate
Apex: acute and often twisted sideways
Color: Upper surface: Chrome Gr. MDP (23-H-12)

Lower surface: Eden Gr. (20-L-9)

Midvein: Lettuce Gr. (20-L-5)

Margin: serrate generally except at basal margins which are crenate and somewhat irregular

### Petiole:

Length: 0.64 inch (1.63 cm)

Color: Lettuce Gr. (20-L-5)

Glands: none
Stipules: none

### Flowers

Since the flowers are imperfect and cannot form fruit, there is no scientific reason for including them in the description of PAC 941.

### Major Uses

The major use of this invention is for rootstock for French prune variety ‘Stanley’, Japanese plum (Prunus salcinia) varieties ‘Blackgold’, ‘Red Beaute’, ‘Golden Globe’, ‘Grand Rosa’, and ‘Son Gold’. In addition, this invention has demonstrated good compatibility with the nectarine and peach (Prunus persica) varieties ‘Summer Grand’, ‘Big Top’, and ‘Alexandra’, as well as the almond varieties (Prunus dulcis) ‘Montayo’, ‘Desmayo Largueta’, ‘Butte’, ‘Monterey’, and ‘Nonpareil’ and apricot varieties.

This invention shows some indication of some resistance to root-knot nematodes (Meloidogyne species), root lesion nematodes (Pratylenchus vulnus), and wet soils.

This invention appears limited to adaptation to heavy amounts of water (water logging) and calcareous soils.

This invention propagates well by in vitro culture techniques.

What is claimed is:

1. A new distinct cultivar of plum almond hybrid exhibiting the following combination of characteristics:
a. superior rooting anchorage when compared to ‘Marianna 2624’ (non patented in U.S.);
b. less propensity to form suckers than most plum rootstocks;
c. aptitude to serve well as a rootstock for plums, peaches, nectarines, almonds and apricots; and
d. outstanding adaptation to limiting soil conditions, especially water logging and calcareous soil.
